(19) United States
(12) Patent Application Publication
(10) Pub. No.: US 2023/0241626 A1
(43) Pub. Date: Aug. 3, 2023

(54) Magnetic Bead Reaction System

(71) Applicant: IDEX Health & Science, LLC, Rohnert Park, CA (US)

(72) Inventors: John Barnacle, Petaluma, CA (US); Eric Beemer, Anacortes, WA (US); Craig Love, Santa Rosa, CA (US); Jamie Bravo, Apopka, FL (US); Carl Sims, Durango, CO (US)

(73) Assignee: IDEX Health & Science, LLC, Rohnert Park, CA (US)

(21) Appl. No.: 18/103,612

(22) Filed: Jan. 31, 2023

Related U.S. Application Data
(60) Provisional application No. 63/304,858, filed on Jan. 31, 2022.

(51) Int. Cl. B03C 1/28; B03C 1/01

(52) U.S. Cl. CPC B03C 1/288; B03C 1/01; B03C 2201/18

(57) ABSTRACT

A magnetic particle suspension system includes a vessel defining a chamber and an opening to the chamber, particles magnetically responsive to a magnetic field disposed in the chamber, and a suspension apparatus for controllably moving the magnetic field along a path having an engagement portion in which the magnetic field effects a magnetic response in the particles, wherein the suspension apparatus includes a motor, an arm movably driven by the motor, and a magnet supported by the arm for movement along the path, wherein the motor rotatably drives the arm about a drive axis so that the magnet travels along a circumaxial path about the drive axis.

### FIELD OF THE INVENTION

The present invention relates to reaction and separations systems generally, and more particularly to reaction systems that use magnetic particles to prepare, separate, chemically modify, concentrate, or otherwise isolate target molecules from a mixture.

### BACKGROUND OF THE INVENTION

Sub-micrometer magnetic particles were initially developed for certain specific chemical applications, but more recently have found use as agglomerations forming micron-sized particles with various surface chemical modifications. These particles are sometimes referred to as magnetic beads or simply “beads”. The particles may be monodisperse, and may have selected functionalized surface modifications to selectively isolate target molecules or biomolecules from complex mixtures.

Advances in the applications of magnetic particles, surface chemistry, porosity, particle size distribution, and the like are related to the development of various chromatographic media. Some magnetic particles have been developed for biological applications, with surface modifications to perform antibody-antigen types of target molecule capture from mixtures, which require gentle capture of analytes from highly complex biological mixtures, followed by selective release. Many biological assays have been developed using specific surface modifications to magnetic particles to extract, purify, concentrate, or otherwise prepare a sample for use or further analysis. Magnetic particles have also been used to capture biologically important molecules from cell cultures in a production environment.

Magnetic particles have been used in bioreactor systems for capturing target synthetic biomolecules and separating them from the reactor media. In an exemplary system described in U.S. Pat. No. 10,940,485, magnetic particles with attached target molecules are transferred to a separate vessel in which the magnetic particles and target molecules are held stationary using positionable magnets. As is well known in the art, magnets may be applied in close proximity to a vessel containing magnetic particles and bound target molecules in a media in order to hold the magnetic particles and bound target molecules in place while the media is removed from the vessel. Subsequent to media removal, wash cycles may be employed to remove residual media from the particles and bound target molecules. The application of magnets is typically removed during the wash cycles so that the magnetic particles and bound target molecules can fully interact with the wash reagents. Thereafter, a transfer solution may be introduced to the vessel in order to release the target molecules from the magnetic particles. Re-application of a magnetic field to the chamber after introduction of the transfer solution physically separates the magnetic particles from the target molecules, thereby permitting removal of the target molecules with the transfer solution.

Magnetic fields may be applied in connection with magnetic particles for bulk capture and release of target compounds with affinity toward the magnetic particles, or at least a surface thereof. Permanent magnets are a common source of a useful magnetic field for this purpose. The permanent magnets may be secured to a movable apparatus that is controlled to selectively apply and remove the associated magnetic field with respect to the magnetic particles in an adjacent vessel. Specifically, the permanent magnet is moved by the movable apparatus into a position in which its magnetic field interacts with the magnetic particles in a vessel to hold the magnetic particles against a container wall while fluids are introduced to and/or removed from the vessel.

Some conventional systems employ mechanical or fluidic mixing mechanisms to suspend the magnetic particles in the respective fluid media when the magnetic field is not applied. The magnetic particles may have a uniform or non-uniform density, and, in some cases, approximate the density of the fluidic media in which the magnetic particles are suspended. Mechanical mixers such as an impeller or rotating vessel are described in U.S. Application Publication No. 2020/0030816 and U.S. Application Publication No. 2019/0022665. It can be important to achieve a uniform dispersion of the magnetic particles in the fluidic media, particularly where sample aliquots may be withdrawn from the particle solution. Although conventional mixing mechanisms adequately disperse the magnetic particles within the fluidic media, some reactor environments are not compatible therewith. For example, mechanisms that rely upon movement of the vessel to achieve magnetic particle mixing are incompatible with applications in which the vessel must be stationary, or connected fixtures to the vessel cannot be rotated or otherwise moved. Conventional systems also do not provide for an ability to modify the mixing speeds relative to particle:fluidic media density and viscosity differences. Moreover, conventional systems do not provide for an ability to change the velocity of the magnetic field relative to the magnetic particles. Conventional systems are limited in their ability to compensate for changes in particle and/or solution variations, which limits optimization of particle manipulation in changing solvent compositions.

### SUMMARY OF THE INVENTION

By means of the present invention, a changeable magnetic field may be controllably applied to a magnetic particle contact system to effect desired movement and non-movement to the magnetic particles. In particular, the magnetic field itself may be moved relative to a stationary vessel containing the magnetic particles in a manner that effects a magnetic response in the magnetic particles. The controlled movement of the magnetic field through the particle-containing chamber of the vessel induces a controlled movement or non-movement of the magnetic particles.

In one embodiment, a magnetic particle suspension system includes a vessel that defines a chamber and an opening to the chamber, particles magnetically responsive to a magnetic field, and a suspension apparatus for controllably moving the magnetic field through the chamber to thereby magnetically control the particles when the particles are disposed in the chamber. The suspension apparatus includes a motor, an arm coupled to the motor, and a magnet coupled to the arm, wherein the motor rotatably drives the arm about a drive axis so that the magnet travels along a circumaxial path about the drive axis.

In some embodiments, the magnetic field generated by the magnet effects a magnetic response in the magnetic particles when in an engagement portion of the circumaxial path. The magnetic response may be attraction to induce movement of the particles. The magnetic response may preferably draw the particles towards or against a wall in or defining the chamber.

The engagement portion of the circumaxial path may be in sufficient proximity to the chamber of the vessel for the magnetic field generated by the magnet to effect the magnetic response in the particles. Additionally, the magnetic field generated by the magnet may not effect the magnetic response in the particles when in a non-engagement portion of the circumaxial path.

The suspension apparatus may include a plurality of arms coupled to the motor and at least one magnet coupled to each of the arms. First and second ones of the arms may extend radially oppositely from the drive axis. The magnets may be drivable about the drive axis along respective circumaxial paths, wherein a first magnetic field generated by a first one of the magnets may effect a magnetic response in the particles when in an engagement portion of its respective circumaxial path, and a second magnetic field generated by a second one of the magnets may effect a magnetic response in the particles when in an engagement portion of its respective circumaxial path. The magnetic response may be attraction to induce movement of the particles. The magnetic response may draw the particles towards or against a wall in or defining the chamber. The first and second magnetic fields may not overlap, and the first circumaxial path may be adjacent to a first side of the vessel, and the second circumaxial path may be adjacent to a second side of the vessel opposite to the first side.

The motor may be a variable-speed motor that is controllable to incrementally rotate a drive member about the drive axis. The arm may be coupled to the drive member. In some embodiments, the motor is a stepper motor with an incremental rotation of the drive member about the drive axis being one degree.

A method for suspending particles in a vessel includes loading particles responsive to a magnetic field into a chamber defined by a vessel, and optionally loading a fluid volume into the chamber. A first magnetic field is applied to effect a magnetic response in the particles, and a second magnetic field is applied to effect a magnetic response in the particles. Prior to or subsequent to applying the second magnetic field, the first magnetic field may be adjusted to change the first magnetic response in the particles.

In some embodiments, the first magnetic field is removed prior to applying the second magnetic field. The first magnetic response may be different from the second magnetic response.

The first magnetic field may be applied from a first side of the vessel, and the second magnetic field may be applied from a second side of the vessel. In some embodiments, the first side of the vessel is opposite from the second side of the vessel.

The first and second magnetic responses typically include attraction. In some embodiments, the first and second magnetic responses each draw the particles towards or against a respective wall in or defining the chamber. At least a portion of the fluid volume may be removed from the chamber while at least one of the first and second magnetic fields is applied to the particles. A second fluid volume may be loaded into the chamber while the at least one of the first and second magnetic fields are applied to the particles.

The first and second magnetic fields may be applied in a manner to distribute and suspend the particles within the fluid volume. In some embodiments, the particles may be substantially uniformly distributed and suspended within the fluid volume.

### BRIEF DESCRIPTION OF THE DRAWINGS

FIG. 1 is a schematic illustration of a magnetic particle suspension system of the present invention.

FIG. 2A is a schematic illustration of the magnetic particle suspension system of FIG. 1.

FIG. 2B is a schematic illustration of the magnetic particle suspension system of FIG. 1.

FIG. 3A is an illustration of a vessel portion of a magnetic particle suspension system of the present invention.

FIG. 3B is a cross-sectional view of FIG. 3A taken along cut line 3B-3B

FIG. 4A is an edge view of a magnet portion of a magnetic particle suspension system of the present invention.

FIG. 4B is an illustration of a magnetic field distribution of the magnet portion illustrated in FIG. 4A.

FIG. 4C is an illustration of a magnetic field gradient of the magnet portion illustrated in FIG. 4A.

FIG. 5 is a schematic illustration of a magnetic particle suspension system of the present invention.

FIG. 6A is a schematic illustration from a cross-sectional top view of a magnetic particle suspension system of the present invention as taken along cut line 6A-6A in FIG. 6B.

FIG. 6B is a schematic illustration from a side view of a magnetic particle suspension system of the present invention.

FIG. 7A is a schematic illustration from a cross-sectional top view of a magnetic particle suspension system of the present invention as taken along cut line 7A-7A.

FIG. 7B is a schematic illustration from a side view of a magnetic particle suspension system of the present invention.

FIG. 8A is a schematic illustration from a cross-sectional top view of a magnetic particle suspension system of the present invention as taken along cut line 8A-8A.

FIG. 8B is a schematic illustration from a side view of a magnetic particle suspension system of the present invention.

FIG. 9 is an overview flow diagram of an example application of a magnetic particle suspension system of the present invention.

FIG. 10 is a flow diagram of a portion of the example application described in FIG. 9.

FIG. 11 is a flow diagram of a portion of the example application described in FIG. 9.

FIG. 12 is a flow diagram of a portion of the example application described in FIG. 9.

FIG. 13 is a schematic illustration of a magnetic particle suspension system of the present invention.

FIG. 14 is a chart overlaying chromatographic data obtained from glycan capture and release from magnetic beads using a magnetic mixing process verses a manual vortexing process.

### DETAILED DESCRIPTION OF THE INVENTION

The objects and features enumerated above together with other objects, features, and advances represented by the present invention will now be presented in terms of detailed embodiments described with reference to the attached drawing figures. Other aspects of the invention are recognized as being within the grasp of those having ordinary skill in the art.

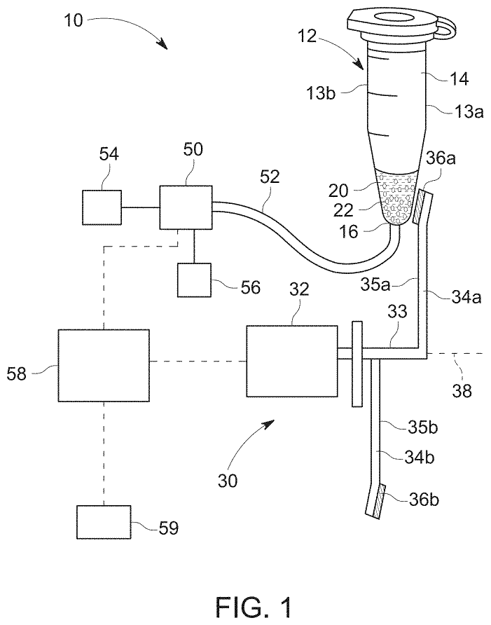

FIG. 1 is a schematic illustration of a magnetic particle suspension system 10 which can be used to magnetically manipulate magnetic particles in a vessel to prepare, separate, chemically modify, concentrate or otherwise isolate target molecules from a mixture. Suspension system 10 includes a vessel 12 defining a chamber 14 and an opening 16 to the chamber. Vessel 12 is configured to receive in chamber 14 a fluid 20 and particles 22 that are magnetically responsive to a magnetic field. Fluid 20 may comprise one or more liquids, gases, solutes, solvents, dispersions, suspensions, solid particles, gels, and the like. Particles 22 may be selectively dispersed, suspended, mixed, or separated in or from fluid 20, as will be described in greater detail hereinbelow.

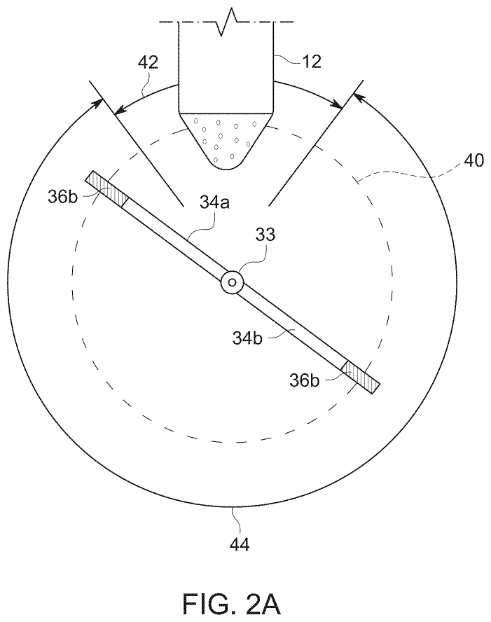

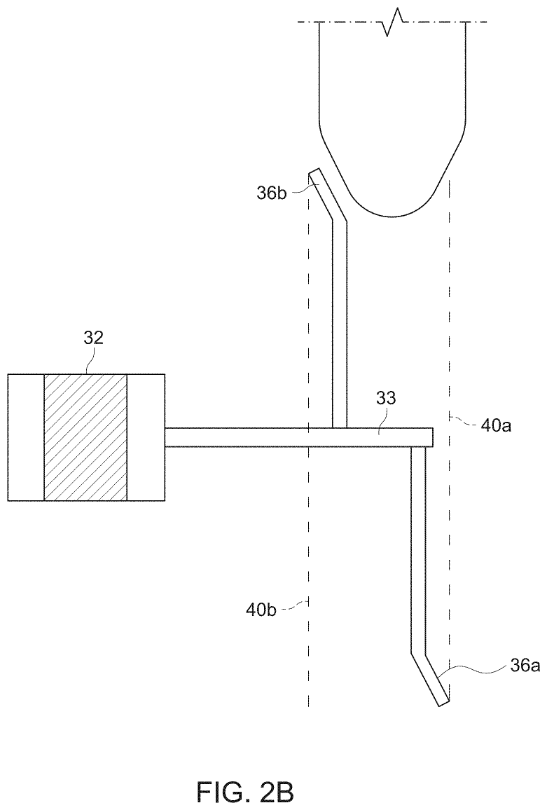

Suspension system 10 further includes a suspension apparatus 30 for controllably moving a magnetic field through chamber 14 to thereby control particles 22 in chamber 14. Suspension apparatus 30 includes a motor 32, a driven member 33, arms 34a, 34b coupled to driven member 33, and a magnet 36a, 36b coupled to a respective arm 34a, 34b. Motor 32 preferably drives driven member 33 about a drive axis 38 so that arm 34a, 34b is correspondingly driven about drive axis 38. As schematically illustrated in FIGS. 2A and 2B, magnets 36a, 36b may be driven by motor 32 along a circumaxial path 40, respectively circumaxial paths 40a, 40b, a portion of which may be in close proximity to vessel 12.

A fluid pump 50 is fluidically connected to chamber 14 through opening 16 by a fluid channel 52. In some embodiments, fluid channel 52 may be a flexible hose made from a polymer or other material that is inert to fluid 20. Pump 50 may be adapted to selectively fill and drain chamber 14 through opening 16, and in connection with reservoirs 54, 56. Opening 16 of chamber 14 may be selectively filled with a gas bubble 21 to prevent fluid from draining out of chamber 14, and to separate the fluid 20 within chamber 14 from the fluid within fluid channel 52. In some embodiments, reservoirs may store wash fluids, eluents, reagents, transfer fluids, storage fluids, magnetic particles, target molecules, waste, and the like. A controller 58 is preferably communicatively linked to fluid pump 50 and motor 30 for automatically controlling the operation thereof, and may additionally be communicatively linked to one or more sensors for receiving signals indicative of various conditions of suspension system 10.

Particles

As described herein, magnetically-responsive particles may be used in a variety of applications, such as reactions, purifications, separations, exchanges, isolations, and the like. Typically, the magnetically-responsive particles are used to permanently or temporarily bind target substances. The binding mechanism may include covalent, non-covalent, electrostatic, hydrogen, Van der Waals, or other suitable forces. Various magnetically-responsive particles, particle shapes, particle sizes, particle size distributions, agglomerations, and particle surface treatments may be used to accomplish the desired process. For the purposes hereof, a particle magnetically responsive to a magnetic field, or a magnetically-responsive particle, is a particle or an agglomeration or cluster of particles that possess magnetic properties or are capable of being magnetized in the presence of a magnetic field. Such particles may also be referred to as “magnetic beads” or “superparamagnetic particles”. The term “magnetic” is intended to include any element that is magnetic, paramagnetic, or ferromagnetic.

Example particles useful in the magnetic particle suspension system of the present invention include iron oxides, such as magnetite (Fe₃O₄), which exhibit magnetic behavior only in the presence of an external magnetic field. This property is dependent upon the small size of the particles, and enables the beads to be separated in suspension, along with anything they are bound to. Such particles do not typically attract each other outside of a magnetic field, so they may be used without concern for unwanted clumping. Types of particles include carboxylate-modified magnetic beads, amine-blocked magnetic beads, oligo(dT)-coated magnetic beads, streptavidin-coated magnetic beads, streptavidin-blocked magnetic beads, protein A/G magnetic beads, silica-coated magnetic beads, and magsepharose.

The magnetically-responsive particles may range in size, shape, and density. As will be described in greater detail hereinbelow, system 10 is preferably adapted to accommodate various magnetically-responsive particles and particle blends, along with various suspension fluid densities and viscosities to achieve desired dispersions of the particles within the suspending fluid. In particular, the magnetically-responsive particles may have a density that is greater, less, or substantially equal to the suspending fluid, as well as various hydrodynamic diameters, and the suspending fluid may be selected for its chemical properties rather than its physical properties, thereby requiring system 10 to adjust a rotational speed for driven member 33 to effect the desired magnetically-driven movement of the magnetically-responsive particles in chamber 14. The magnetically-responsive particles may typically range in size from 1 nm to 5 μm, preferably from 1 nm to 3 μm, and more preferably from 1 nm to 100 nm. Non-spherical magnetically-responsive particles may exhibit a hydrodynamic diameter of from 1 nm to 5 μm, preferably from 1 nm to 3 μm, and more preferably from 1 nm to 100 nm. The magnetically-responsive particles may be spherical, non-spherical, rod-like, plate-like, symmetrical, or asymmetrical in shape.

### Vessel

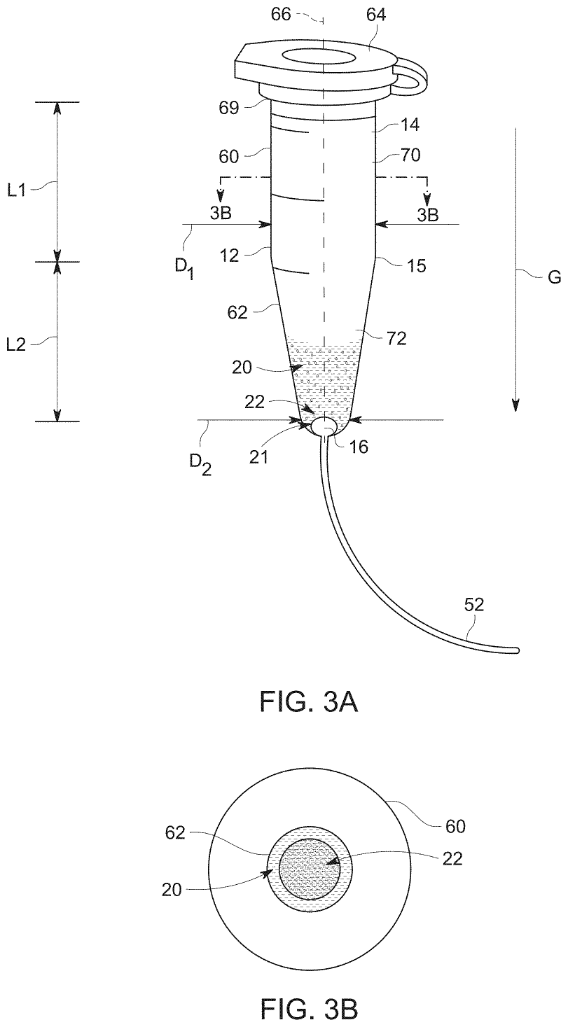

An example vessel 12 useful in magnetic particle suspension system 10 is illustrated in FIG. 3. Vessel 12, in the illustrated embodiment, is a conical microcentrifuge tube such as those commercially available from, for example, ThermoFisher Scientific, modified to include opening 16 for the introduction and removal of materials to and from chamber 14 through fluid channel 52. Vessel 12 includes an exterior wall 15 defining chamber 14. Exterior wall 15 has a cylindrical region 60 and a circular conical region 62 that narrows from cylindrical region 60 to opening 16. Cylindrical region 60 of exterior wall 15 terminates in a second opening that may be removably closed with cap 64. In some embodiments, vessel 12 is fabricated from one or more polymers which are preferably inert to the fluids loaded into chamber 14. Example polymers useful for the manufacture of vessel 12 include polypropylene (PP) and polyethylene terephthalate (PET). However, vessel 12 may be made from one or more of a variety of suitable materials for the intended applications of system 10. Vessel 12 may have a volumetric capacity suitable for the intended application. In some embodiments, vessel 12 may have a volumetric capacity in chamber 14 of between 1-2 mL.

Applicant has found that the shape of chamber 14, as well as its operating orientation relative to gravity, may assist in the desired magnetic interaction between particles 22 and magnet 36a, 36b. As shown in FIG. 3, a central axis 66 of chamber 14 extending through opening 16 and the upper opening may preferably be oriented along a gravitational vector direction “G”. In this manner, gravitational forces will urge particles 22 toward a portion of chamber 14 defined by conical region 62 of exterior wall 15. Moving magnetic gradients from magnet 36a, 36b as it is driven along circumaxial path 40 cause particles 22 disposed at that region of chamber 14 to move in a swirling pattern that randomizes as magnet 36a, 36b sequentially imparts respective magnetic fields to particles 22. Such motion greatly improves dispersion of particles 22 in fluid 20 over conventional systems, and leads to more consistently uniform particle suspensions.

In some embodiments, chamber 14 has a cylindrical portion 70 associated with cylindrical region 60 of exterior wall 15, and a circular conical portion 72 associated with circular conical region 62 of exterior wall 15. In an example embodiment, an Eppendorf® 1.5 mL microcentrifuge tube from Sigma-Aldrich, modified with opening 16 to receive tubing 52, was utilized as the vessel 12. It is contemplated, however, that other sizes and shapes for vessel 12 may be useful in the apparatus of the present invention. Cylindrical portion 70 of chamber 14 may have a diameter D₁ (inner diameter of vessel 12) and a length L₁, as defined between a transition 68 between cylindrical region 60 and conical region 62 and a second end 69 of vessel 12. Circular conical portion 72 of chamber 14 may have a narrowing diameter from D₁ to D₂ at first end 67 of vessel 12.

### Magnet

For the purposes hereof, the term “magnet” is intended to mean one or more magnetic field generating elements. In some embodiments, each of the magnets 36a, 36b is a permanent magnet that is configured to generate its own persistent magnetic field. Each magnet may, in some embodiments, generate a field strength of less than 1000 Tesla/m, and more preferably between about 0.05 and 150 Tesla/m. Applicant has found that this range of field strength is important to the specific magnetically-induced movement to particles 22 in fluid 20 described herein, while also being capable of drawing particles 22 against an interior surface of exterior wall 15, or against a surface of an internal wall in chamber 14.

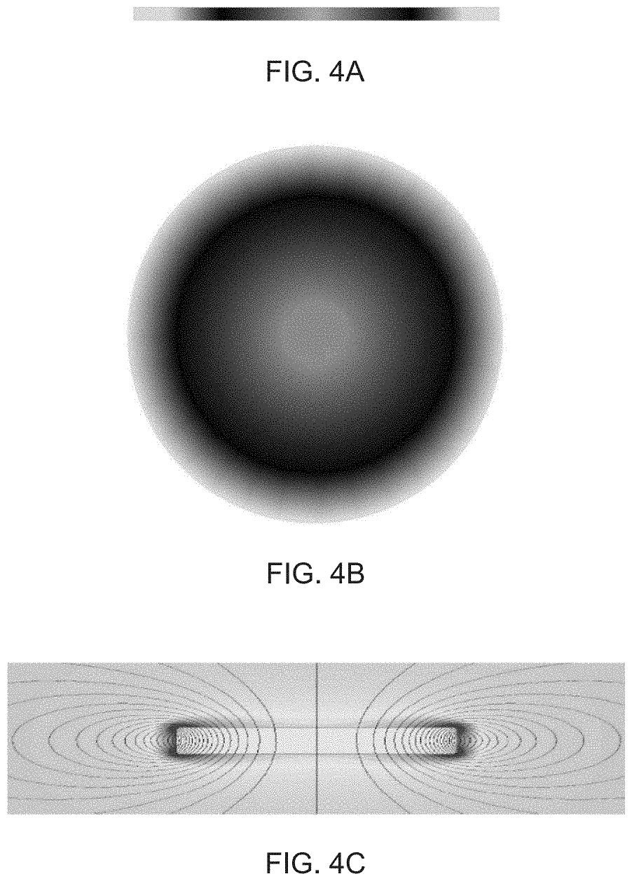

In an example embodiment, each magnet 36a, 36b may comprise a type N52 disc magnet that is axially polarized with 39 Tesla/meter edge field strength and 1.13 Tesla/m center field strength, and having a diameter of 32 mm and a thickness of 3 mm. A magnetic field generated by the example magnet 36a, 36b is illustrated in FIGS. 4A-4C, with the edge magnetic field illustrated in FIG. 4A, the face field distribution illustrated in FIG. 4B, and the field gradient illustrated in FIG. 4C. Applicant has determined that disc-shaped magnets perform well to induce the desired movement to particles 22 in chamber 14. It is contemplated, however, that other magnet shapes could be substituted for the disc-shaped magnets described herein if testing data evidenced similar performance in the inducement of movement to particles 22. It is further contemplated that magnets 36a, 36b may be selected for required magnetic field strength, magnetic field shape, and magnetic field size relative to chamber 14.

In other embodiments, magnets 36a, 36b may be constructed of one or more electromagnets configured to generate a magnetic field when supplied with electrical current. The electromagnets may be adapted to generate a variable intensity magnetic field responsive to an electrical current input.

### Motor

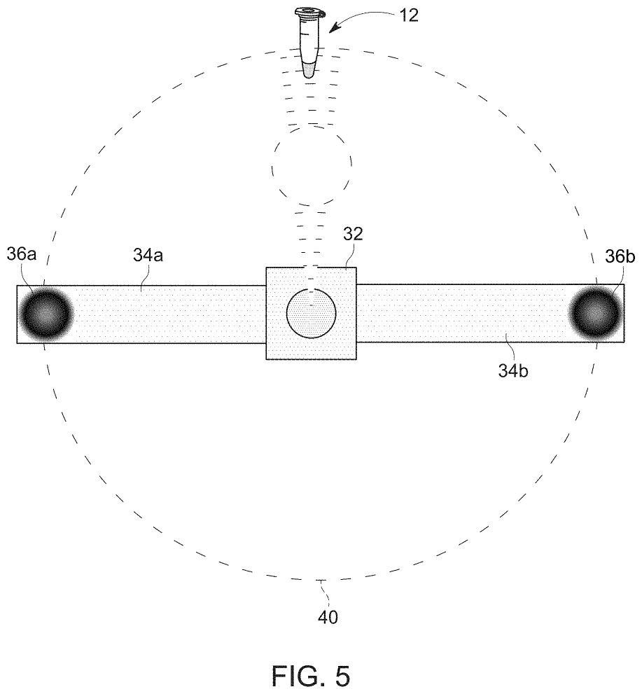

Motor 32 may be any motor suitable to controllably generate rotational motion to driven member 33. Motor 32 is preferably a variable-speed, reversible motor that can be automatically controlled by controller 58. More preferably, motor 32 is an electric stepper motor having a positional accuracy of one part in at least 360 per revolution, preferably one part in at least 1000 per revolution, and more preferably one part in at least 4000 per revolution. A feature of a stepper motor that is useful in the present invention is that it may be configured to rotate at various controlled rates, and to stop at any selected point in its rotation. As schematically illustrated in FIG. 5, motor 32 drives arms 34a, 34b that carry respective magnets 36a, 36b circumaxially about drive axis 38 along respective circumaxial paths, schematically illustrated by dashed line 40. As should be understood from the example embodiment illustrated in FIGS. 1 and 2, however, each magnet 36a, 36b may travel along its own circumaxial path about drive axis 38. Each dash of dashed line 40 may represent a stop point for stepper motor 32. For example, controller 58 may be programmed to operate motor 32 to incrementally rotate driven member 33 in 1 degree increments about drive axis 38. In this manner, the magnetic field generated by magnets 36a, 36b may be selectively positioned relative to chamber 14 to have the desired effect or lack of effect on particles 22.

Controller 58 may desirably operate motor 32 by being responsive to an input. In some embodiments, the input may include a signal from a position sensor indicating the position of magnets 36a, 36b and/or arms 34a/34b relative to chamber 14 of vessel 12. An example position sensor is a shaft encoder. Hall effect devices that are triggered by the magnetic field generated by magnets 36a, 36b may sense the position of magnets 36a, 36b along their respective circumaxial paths 40a, 40b. In some embodiments, an elapsed time between position sensor signals may be used to calculate a rotational speed of motor 32 and magnets 36a, 36b. Controller 58 may track steps of a stepper motor 32 to dynamically determine magnet location relative to a sensed position along circumaxial path 40. Controller 58 may be programmed to stop motor 32 to hold magnets 36a, 36b at any of a plurality of positions along their respective circumaxial paths 40a, 40b for a controlled period of time. In some embodiments, controller 58 may hold a magnet 36a, 36b in place adjacent to vessel 12 and chamber 14 for a controlled period of time to correspondingly hold particles 22 in place in chamber 14.

### Arrangement and Operation

One or more arms 34a, 34b may be secured to driven member 33 in order to be driven about drive axis 38. Arms 34a, 34b may be welded, soldered, adhered, fastened, or formed integrally with driven member 33. In some embodiments, a first arm 34a extends radially outwardly from driven member 33 along a first direction, and a second arm 34b extends radially outwardly from driven member 33 along a second direction that is different from the first direction. The first direction may be opposite from the second direction.

Magnetic particle suspension system 10 is arranged to magnetically control particles 22 when the particles 22 are disposed in chamber 14. The magnetic control of particles 22 includes induced movement in a manner that disperses particles throughout a suspending medium, as well as in a manner to hold particles 22 in place, such as against a wall in chamber 14, while material is loaded and/or removed from chamber 14 through opening 16. Particles 22 are magnetically controlled through the application, modification, and removal of one or more magnetic fields generated by magnets 36a, 36b. The magnetic field generated by each magnet 36a, 36b effects a magnetic response in particles 22 when the respective magnet 36a, 36b is within an engagement portion 42 of circumaxial path 40. It is within engagement portion 42 that the magnetic field generated by magnet 36a, 36b is sufficiently strong to effect a magnetic response in particles 22. Outside engagement portion 42, which is non-engagement portion 44 of circumaxial path 40, the magnetic field generated by magnet 36a, 36b is insufficient to effect a magnetic response in particles 22 for the purposes of the present invention. In particular, the magnetic response useful for the purposes of the present invention is a magnetic attraction suitable to induce movement of particles 22.

As aspect of the present invention is the controlled application, modification, and removal of one or more magnetic fields to magnetically-responsive particles 22 in chamber 14. The arrangement of magnets 36a, 36b secured to respective arms 34a, 34b, along with the controlled rotational movement of driven member 33 about drive axis 38 facilitates a unique interaction between magnetic fields and magnetically-responsive particles. Magnet 36a may be secured to arm 34a so as to be brought into proximity to vessel 14 in the engagement portion 42 of circumaxial path 40. In the illustrated embodiment, magnet 36a may be secured to an inner surface 35a of arm 34a so that magnet 36a is positioned between arm 34a and vessel 14 as magnet 36a is driven through engagement portion 42 of circumaxial path 40. A likewise arrangement may be suitable for magnet 36b at surface 35b of arm 34b. The proximity of magnets 36a, 36b to vessel 14 when in engagement portion 42 of circumaxial path 40 facilitates a sufficient interaction of the respective magnetic fields with particles 22 to effect a magnetic response in particles 22. In some embodiments, magnets 36a, 36b may be brought to within 10 mm of vessel 14, preferably within 5 mm of vessel 14, and more preferably within 1 mm of vessel 14 on circumaxial path 40.

As described above, a magnetic response in particles 22 is an attraction toward the respective magnet 36a, 36b that induces corresponding movement in particles 22. The movement of particles 22 is dependent upon several factors, including characteristics of the magnetic field, movement of the magnetic field, characteristics of particles 22, characteristics of any suspending fluid present, and characteristics of chamber 44. Controller 58 may be programmed to accommodate these characteristics in order to adjust the movement of the magnetic field along circumaxial path to accordingly induce the desired movement in particles 22, as well as to permit movement of particles 22 under gravitational forces alone or in combination with applied magnetic fields.

Applicants contemplate several operating conditions in which to control motor 32 to effect desired behavior in particles 22 in chamber 14. The following Table 1 describes example operating conditions, specified by magnetic field, magnetic field location, and field rotational speed.

TABLE 1

| | | | Field | Induced or |
|---|---|---|---|---|
| Operating | Magnetic | Field | Rotational | Permitted Particle |
| Condition | Field | Location | Speed | Movement |
| 1 | A | Engagement | 0 | Toward or against |
| | B | Non- | 0 | chamber wall in |
| | | engagement | | direction of first |
| | | | | side of vessel |
| 2 | A | Non- | 0 | Toward or against |
| | | engagement | | chamber wall in |
| | B | Engagement | 0 | direction of second |
| | | | | side of vessel |
| 3 | A | Engagement | High | Dispersion in low- |
| | B | Non- | High | viscosity fluid |
| | | engagement | | |
| 4 | A | Engagement | Low | Dispersion in high- |
| | B | Non- | Low | viscosity fluid |
| | | engagement | | |
| 5 | A | Non- | 0, low, high | Settling under |
| | | engagement | | gravitational force |
| | B | Non- | 0, low, high | |
| | | engagement | | |

Operating condition 1 controls motor 32 to stop rotational movement at a selected incremental location about drive axis 38 in which magnetic field “A”, associated with magnet 36a, is positioned in engagement region 42, while magnetic field “B”, associated with magnet 36b, is positioned in non-engagement region 44. In some embodiments, operating condition 1 of controller 58 may instruct motor 32 to stop rotational movement at a selected incremental location about drive axis 38 in which magnet 36a is in close proximity to vessel 12, preferably in which magnet 36a is immediately adjacent to or in contact with vessel 12 in order for magnetic field A to effect the greatest magnetic response in particles 22. Operating condition 1 may control motor 32 to a stop condition for a predetermined period of time, preferably sufficient to draw particles 22 against a wall in or chamber 14. Although not expressly illustrated in the drawings, it is contemplated that vessel 12 may include a wall within chamber 14 that does not define exterior wall 15, but instead forms a surface against which particles 22 may be drawn by a magnetic field in, for example, operating condition 1 of controller 58.

With reference to FIGS. 1 and 2, driven member 33 may be controllably rotated about drive axis 38 by motor at the direction of controller 58. Arms 34a, 34b may rotate with driven member 33 about drive axis 38 so that magnet 36a travels along a first circumaxial path 40a into proximity to a first side 13a of vessel 12. Likewise, magnet 36b travels along a second circumaxial path 40b into proximity to a second side 13b of vessel 12. For operating condition 1 of controller 58, therefore, the magnetic field “A” generated by magnet 36a draws particles toward or against a chamber wall in the direction of first side 13a of vessel 12.

Operating condition 2 of controller 58 controls motor 32 to stop rotational movement at a selected incremental location about drive axis 38 in which magnetic field “B” is positioned in engagement region 42, while magnetic field “A” is positioned in non-engagement region 44. In some embodiments, operating condition 2 of controller 58 may instruct motor 32 to stop rotational movement at a selected incremental location about drive axis 38 in which magnet 36b is in close proximity to vessel 12, preferably in which magnet 36b is immediately adjacent to or in contact with vessel 12 in order for magnetic field B to effect the greatest magnetic response in particles 22. Operating condition 2 may control motor 32 to a stop condition for a predetermined period of time, preferably sufficient to draw particles 22 against a wall in or defining chamber 14 in the direction of second side 13b of vessel 12.

Operating conditions 3 and 4 of controller 58 control motor 32 to rotate driven member 33 about drive axis at a “high” or “low” rotational speed, including as magnetic field A travels through engagement region 42 and as magnetic field B travels through non-engagement region 44. Although the specific rotational speed of driven member 33 in any of operating conditions 3-5 is not limited, an aspect of the invention is that controller 58 may be programmed to modify the rotational speed of motor 32/driven member 33 to achieve a desired movement of particles 22 in chamber 14. In many applications, it may be desirable to disperse and even substantially uniformly disperse particles 22 in the suspending fluid 20. Dispersal of particles 22 in suspending fluid 20 may enhance the efficiency and effectiveness of target substance transfer to and from particles 22. Thorough dispersal of particles 22 in suspending fluid 20 may also facilitate consistency of samples taken from the dispersion.

The dispersion of particles 22 in suspending fluid 20 is dependent upon several factors, including the viscosity of the suspending fluid 20, the magnetic characteristics of particles 22, the characteristics of the magnetic field(s) effecting a response in particles 22, and the characteristics of chamber 14. Controller 58 may preferably be programmed with algorithms that permit controller 58 to calculate suitable rotation speeds for motor 32/driven member 33 to desirably disperse particles 22 in suspending fluid 20, including substantially uniformly dispersing particles 22 in suspending fluid 20. The algorithms may take into account the characteristics of chamber 14, including volume, diameters, and the like, as well as the characteristics of magnets 36a, 36b, including magnet shape and magnetic field generated by each of magnets 36a, 36b. The algorithms may further take into account a radius of circumaxial path 40. Variable information pertaining to the specific suspension application may be input to controller through, for example, a user interface 59 such as a computer terminal or graphical user interface of controller 58. The variable information input to controller 58 may include, for example, suspending fluid types, fluid viscosities, fluid volumes, and particle types, particle densities, particle density distributions, particle sizes, particle size distributions, particle hydrodynamic sizes, and particle hydrodynamic size distributions. Such information governs the movement of particles 22 through suspending fluid 20, pursuant to Stoke's Law.

The example operating conditions 3 and 4 represent different controlled rotational speeds for driven member 33 depending upon the viscosity of suspending fluid 20. Motor 32 is preferably capable of adjusting rotational speed and/or reversing rotational direction in response to instructions from controller 58. It has been found that improved mixing/dispersion of particles 22 in a relatively high viscosity suspending fluid 20 may be accomplished with a relatively lower rotational speed, in order to increase the engagement time of a magnetic field within engagement portion 42 of circumaxial path 40. The relatively high fluid viscosity retards particle acceleration rates, wherein a longer engagement time permits the particles 22 to attain sufficient velocities for desired mixing/dispersion. By contrast, improved mixing/dispersion of particles 22 in a relatively low viscosity suspending fluid 20 may be accomplished with a relatively higher rotational speed, in order to increase the frequency of opposing magnetic field interactions with particles 22. The relatively low fluid viscosity can tend to permit particles 22 to move in a clump within suspending fluid 20. As a result, increasing the frequency of exposure to magnetic fields that act to change particle movement directions can aid in dispersing the particles 22.

Operating condition 5 of Table 1 represents an additional degree of particle movement control, wherein particles 22 may be permitted to be acted upon solely by gravitational forces for a controlled period of time. To do so, first and second magnetic fields A, B may be simultaneously positioned in non-engagement portion 44 of circumaxial path 40. Such arrangement of first and second magnetic fields A, B may be sustained for a controlled period of time, depending upon the rotational speed of motor 32/driven member 33. The rotational speed may be equal to or greater than 0, and is preferably adjustable by controller 58.

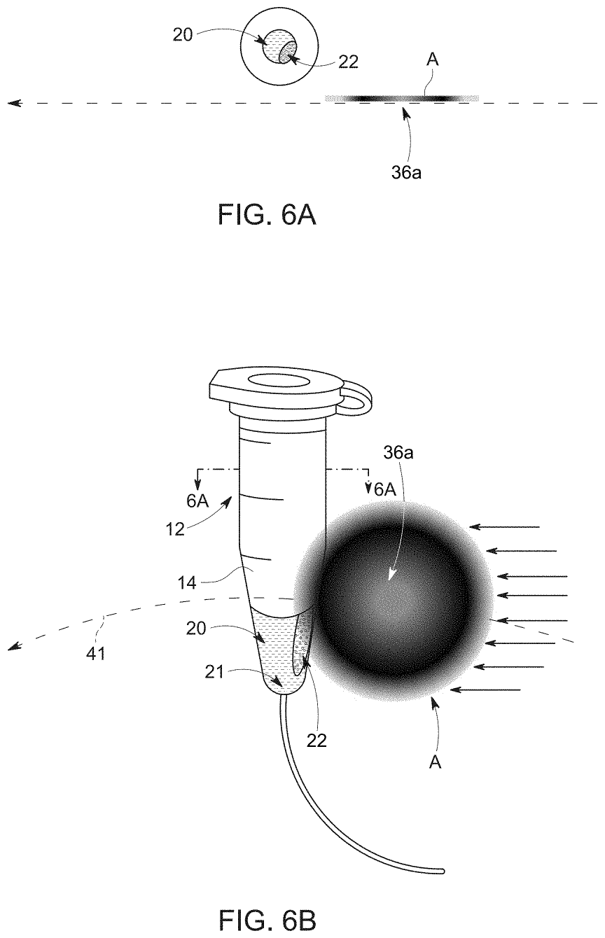

FIGS. 6A-8B illustrate a progression of particle movement in chamber 14 in connection with an arcuate path of a moving magnetic field in proximity to chamber 14. FIG. 6a is a top view of the side perspective view of FIG. 6B, wherein the arcuate path represents a segment of circumaxial path 40 as magnet 36a, with corresponding magnetic field “A”, travels in proximity to vessel 12 in engagement portion 42. The direction of travel of magnet 36a is demonstrated by the arrowhead of arcuate path 41 in both FIGS. 6A and 6B. In the illustrated embodiment, particles 22 are drawn toward and/or against exterior wall 15 at a first side 13a of vessel 12. It has been found by applicant that mixing/dispersal of particles 22 in suspending fluid 20 may be enhanced with the sample being disposed in an inverted circular conical region 72 of chamber 14. It is believed that the relationship of an arcuate path of a moving magnetic field past the magnetically-responsive particles in circular conical region 72 creates a randomized swirling fluid movement that assists in distributing particles 22 throughout suspending fluid 20. Moreover, particles 22 magnetically responsive to a slowly moving or stopped magnetic field may be more grouped together when magnetically held against a tapered wall of the circular conical region 72. This characteristic can aid in holding particles 22 in place while exchanging fluid in chamber 44.

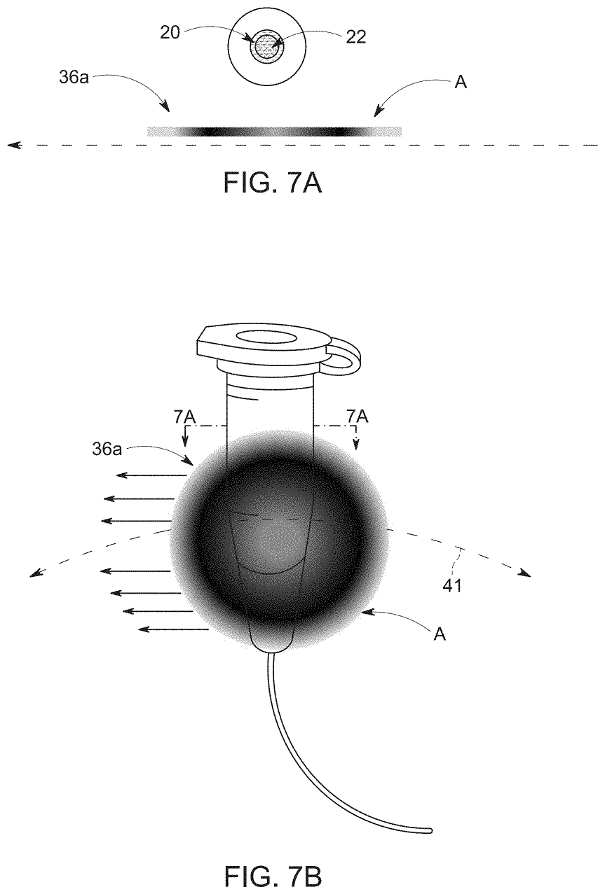

FIGS. 7A-7B illustrate magnet 36a, field A, at a position most proximate to vessel 12. Particles 22 continue to be drawn toward magnetic field A. In some embodiments, the circular conical configuration of conical region 72 may cause particles 22 to be drawn toward a first end of chamber 14 when magnetic field A is in the position illustrated in FIGS. 7A-7B.

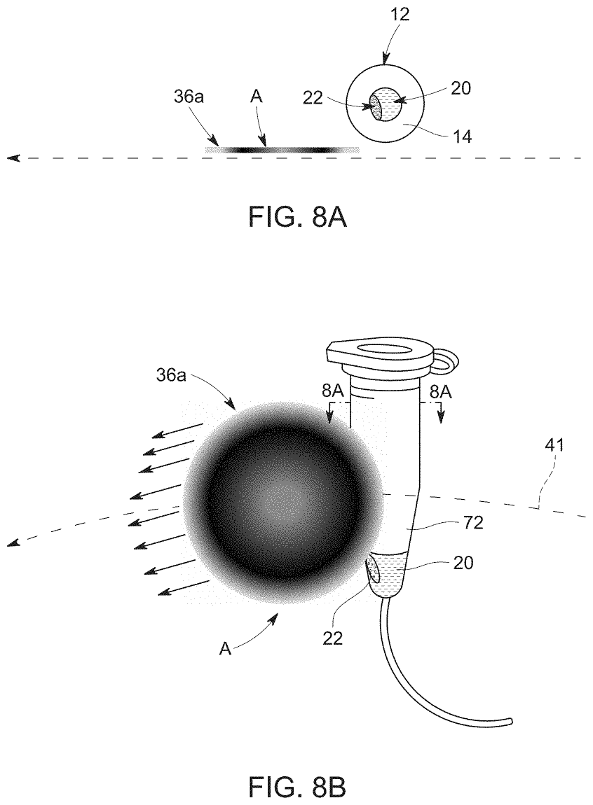

FIGS. 8A-8B illustrate the continued movement of particles 22 to follow moving magnetic field A along arcuate path 41.

Although not expressly illustrated in FIGS. 6A-8B, it should be understood that the passing of first magnet 36a by first side 13a of vessel 12 is preferably followed by the passing of second magnet 36b by second side 13b of vessel 12. The passing of first magnet 36a by first side 13a preferably induces a first rotational motion to particles 22 in fluid 20, in part due to the configuration of conical region 72 of chamber 14. The induced first rotational motion of particles 22 may impart a drag rotational velocity to the fluid, which may continue for a period of time after the magnetic field of first magnet 36a is removed from chamber 14. The passing of second magnet 36b by second side 13b, which may be opposite to first side 13a, induces a second rotational motion to particles 22 in fluid 20. The second rotational motion may preferably be different than the first rotational motion, and may include velocity component vectors that are opposite from the velocity component vectors of the first rotational motion. The same is true for an imparted drag rotational velocity to the fluid, which may continue for a period of time after the magnetic field of second magnet 36b is removed from chamber 14. The inducement of changing rotational motions to particles 22 results in a turbulence that encourages mixing and dispersion of particles 22 throughout the fluid volume. The turbulence promotes the desired chemical interactions between the magnetically-responsive particles and the fluid, such as for substance transfer operations.

In the illustrated embodiment, a valve in the form of a gas bubble 21 may be employed at opening 16 to prevent unwanted drainage of the contents of chamber 14, and to separate the contents of chamber 14 from the contents of fluid channel 52. The gas bubble valve 21 may be “closed” by establishing the gas bubble at opening 16 with an intentional operation of fluid pump 50 in combination with one or more fluid reservoirs 54, 56. Once the gas bubble is formed in fluid channel 52, pump 50 may be operated to push the bubble with fluid through fluid channel 52 to or in proximity to opening 16. The gas bubble is preferably of sufficient volume to fill a cross-section of fluid channel 52 or opening 16 in order to substantially prevent material from passing through a valve region established by the gas bubble. Gas bubble valve 21 is preferably effective in preventing the passage of fluid 20 and/or particles 22 therethrough when deployed at opening 16 or in fluid channel 52. In some embodiments, the gas bubble forming gas bubble valve 21 is hydrophobic to assist in preventing the passage of fluid 20 and wetted particles 22. Preferably, gas bubble valve 21 is positioned at opening 16, or within fluid channel 52 at opening 16, so that fluid 20 and particles 22 remain in a magnetically active region 72 of chamber 14 The gas bubble valve 21 may be “opened” to permit drainage and/or filling of chamber 14 by withdrawing the fluid in fluid channel 52 with pump 50 to an extent to which the gas bubble is drawn into a fluidic valve or venting apparatus.

### Example

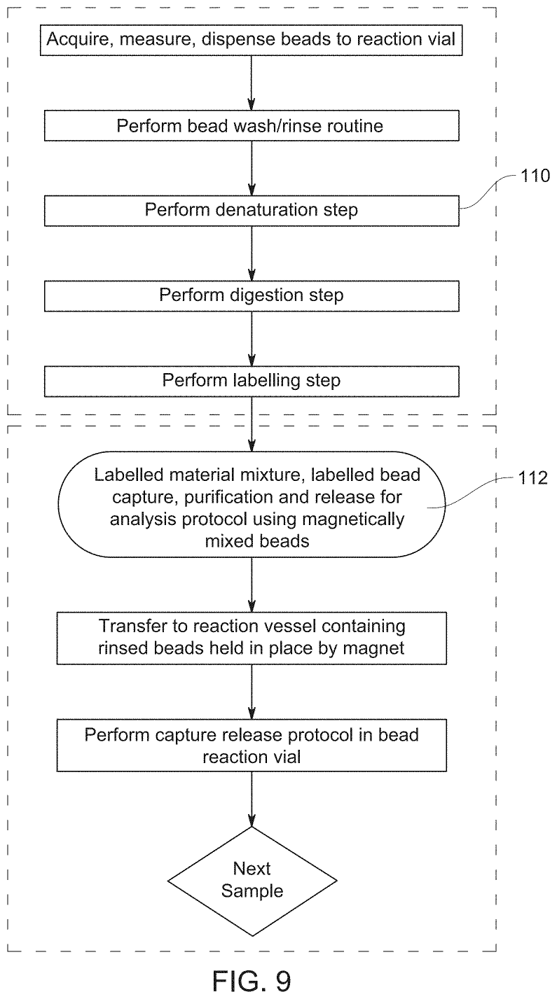

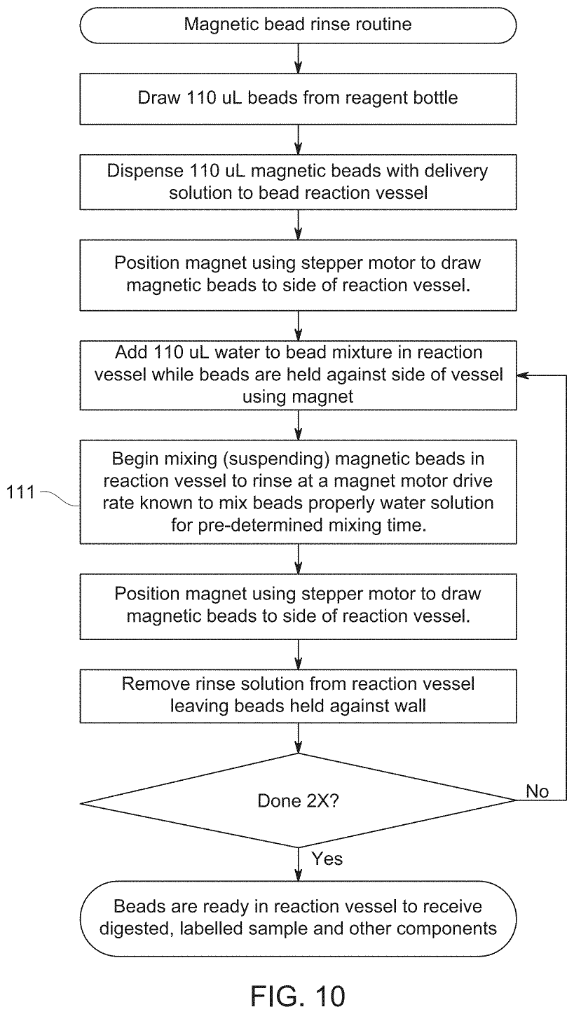

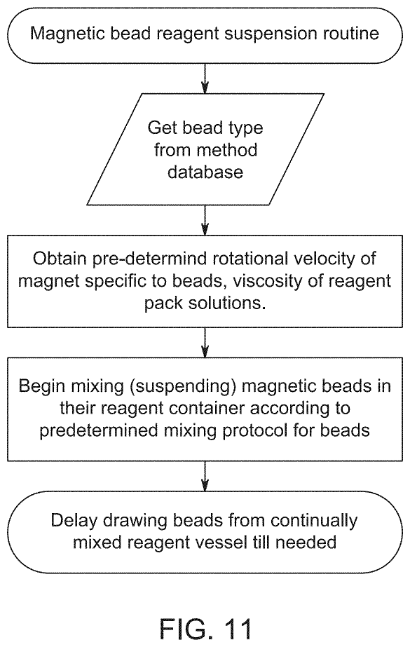

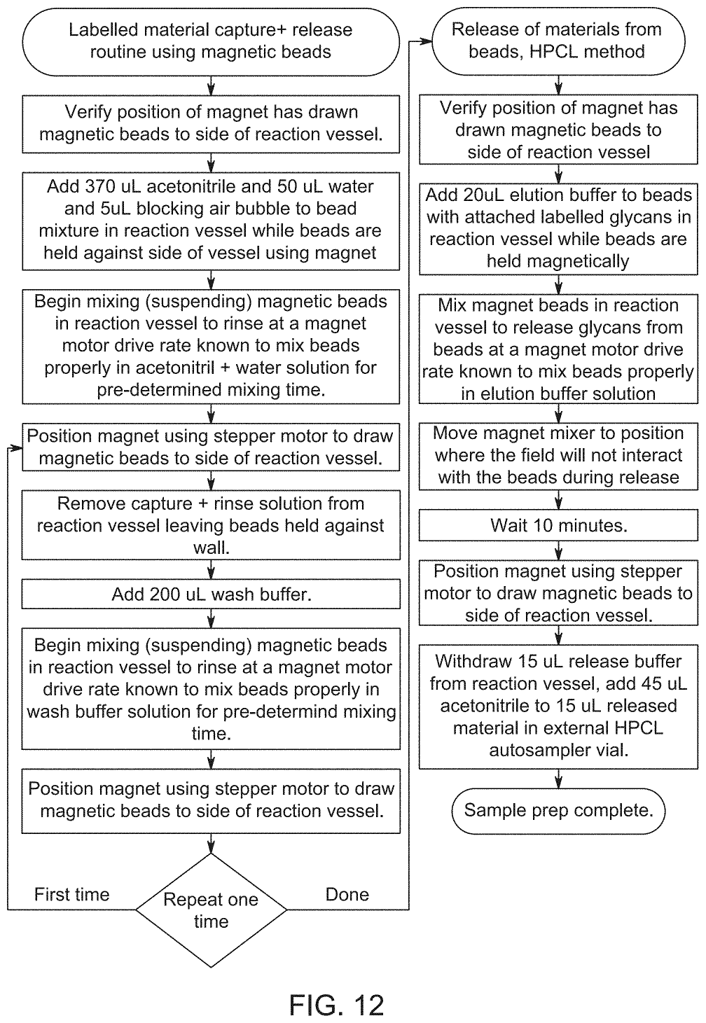

The magnetic particle suspension system of the present invention may be used in a variety of applications. An example application is the purification of n-glycan and labeling of glycoproteins. An overview flow diagram of this example application is illustrated in FIG. 9, and the subset processes of magnetic bead rinse/wash routine 110 (FIG. 10), magnetic bead suspension routine 111 (FIG. 11), and labelled bead capture and release routines (FIG. 12) are also illustrated. It should be understood that the process steps, and specific materials, volumes, and times described in the drawings are merely exemplary of various techniques that may be used in the suspension system of the present invention.

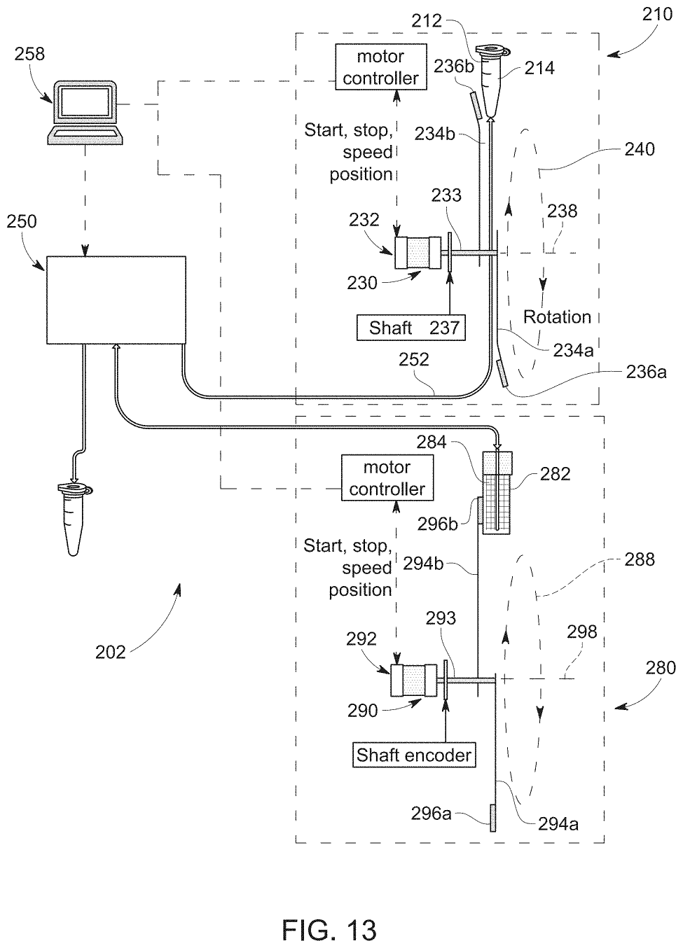

A system 202 for labeling glycoproteins is schematically illustrated in FIG. 13, and generally includes a reaction suspension system 210 and a supply suspension system 280 controlled by controller 258 and coordinating with a fluid pump and valve apparatus 250. Reaction suspension system 210 is analogous to suspension system 10, with a vessel 212 defining a chamber 214, and a suspension apparatus 230 for controllably moving a magnetic field through chamber 214. Suspension apparatus 230 includes a motor 232, a driven member 233, arms 234a, 234b coupled to driven member 233, and magnets 236a, 236b coupled to respective arms 234a, 234b. Motor 232 drives driven member 233 about drive axis 238 to correspondingly drive magnets 236a, 236b along respective circumaxial paths 240a, 240b. Suspension apparatus 230 also includes a sensor, such as a shaft encoder 237, to aid in the incremental operation of motor 232.

Supply suspension system 280 may be arranged substantially similarly to reaction suspension system 210, with the purpose of supply suspension system 280 being to supply consistent and substantially uniform dispersions of magnetic particles in a reagent solution through fluid pump and valve apparatus 250 to reaction suspension system 210. It is contemplated that reaction suspension system 210 may require known and consistent volumes of magnetic particles. To do so, supply suspension system may be operated to maintain magnetic particles in a substantially uniform dispersion in a supply reagent, wherein delivery of a known volume of the supply reagent from reagent vessel 282 contains a known and consistent amount of magnetic particles. Suspension apparatus 290 controllably moves a magnetic field through a chamber 284 defined by reagent vessel 282, and includes a motor 292, a driven member 293, arms 294a, 294b coupled to driven member 293, and magnets 296a, 296b coupled to respective arms 294a, 294b. Motor drives driven member 293 about drive axis 298 to correspondingly drive magnets 296a, 296b along respective circumaxial paths 288a, 288b.

Controller 258 may be configured to communicate with respective motor controllers for motors 232 and 292 in order to independently control their operation. In some embodiments, motor 232 may be operated in selected ones of several different conditions as described above with respect to motor 32. Motor 292 may also be operated in selected ones of several different conditions. Typically, however, motor 292 may be operated to maintain a substantially uniform dispersion of magnetic particles in reagent. Such operation may include continuous rotation of driven member 293 about drive axis 298 at various rotational speeds suitable to accommodate various reagent viscosities that affect particle dispersion.

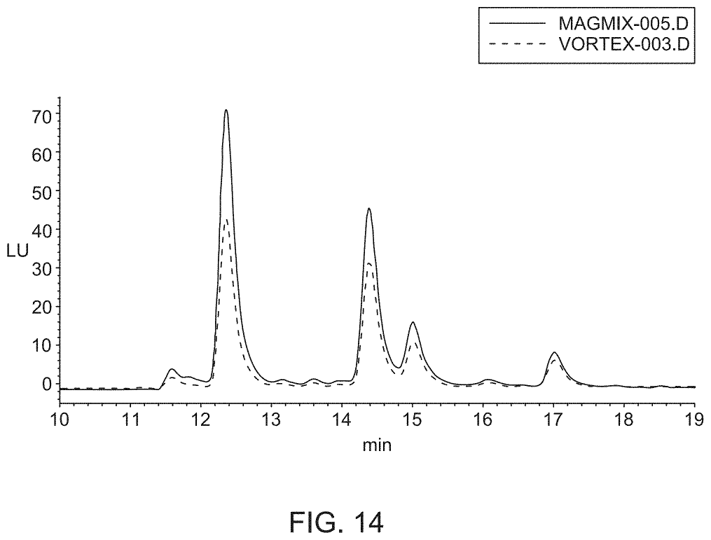

FIG. 14 illustrates chromatographic data acquired from a SILuLite SigmaMab universal antibody standard (Sigma-Aldrich) processed with a GlycanAssure assay kit from ThermoFisher Scientific for released N-glycans captured on GlycanAssure magnetic beads. The chart of FIG. 14 shows a chromatographic data overlay with a first trial (−003.D) performing the glycan capture with manual vortexing, and a second trial (−005.D) performing the glycan capture with the magnetic particle mixing of the present invention. The overlaid chromatographic profiles show the relative N-glycan capture efficiency on the magnetic beads using the two different processes.

A 40 μL sample of 1 mg/mL SigmaMab recombinant human IgG1 sample was manually processed through the Denaturation, Digestion, and Labeling steps. The labeled sample was then split into two equal 30 μL portions. One of the labeled samples (−005.D) and a 50 μL aliquot of magnetic beads were introduced to the reaction cell for the “Bind Beads” step, and the sample/bead combination was mixed by operating the suspension apparatus at 90 rpm while adding 375 μL of acetonitrile (ACN) via the lower tubing connection. The suspension apparatus was then operated to capture the magnetic beads against the reaction chamber wall to isolate the magnetic beads from the liquid while the ACN was removed. A 200 μL aliquot of wash buffer was then added to the reaction chamber for re-suspension of the magnetic beads through operation of the suspension apparatus, and transfer to a 1.5 mL microcentrifuge tube to complete the manual wash and elution steps. The other labeled sample (−003.D) was processed manually following the GlycanAssure workflow for 1.5 microcentrifuge tubes.

Tables 2 and 3 below describe the areas under each chromatographic peak, with Table 2 showing the peak areas for the chromatographic data obtained from the sample processed through the magnetic mixing of the present invention, as well as the relative areas for signal 1 (magnetic mixing) and signal 2 (manual mixing). The relative areas demonstrate increased peak areas for the magnetic mixing method, which is indicative of more captured glycans.

TABLE 2
Magnetic Mixing (−005.D)

| | Time | Area | Height | Relative Area |
|---|---|---|---|---|
| Peak # | (min) | (LU*sec) | (LU) | (Sig1/Sig2) |
| 1 | 11.590 | 34.276 | 3.324 | 1.33 |
| 2 | 12.354 | 1123.878 | 70.561 | 1.65 |
| 3 | 14.386 | 730.772 | 45.051 | 1.42 |
| 4 | 15.005 | 283.870 | 15.606 | 1.40 |
| 5 | 17.009 | 152.956 | 8.675 | 1.29 |

TABLE 3
Manual Vortexing (−003.D)

| | Time | Area | Height | Relative Area |
|---|---|---|---|---|
| Peak # | (min) | (LU*sec) | (LU) | (Sig2/Sig2) |
| 1 | 11.590 | 25.675 | 2.240 | 1.00 |
| 2 | 12.354 | 680.297 | 43.220 | 1.00 |
| 3 | 14.386 | 513.251 | 31.610 | 1.00 |
| 4 | 15.005 | 202.222 | 11.236 | 1.00 |
| 5 | 17.009 | 118.628 | 6.763 | 1.00 |

The purpose of this experiment was to compare the glycan capture efficiency using the suspension apparatus of the present invention in comparison to conventional vortexing. It is believed that, because the magnetic mixing operation results in lower shear rates on the magnetic beads, and the oscillating magnetic field eliminates the “clumping” issue often observed when vortexing the mixtures. The data verifies that the magnetic mixing results in improved glycan capture across all glycan species.

The invention has been described herein in considerable detail in order to comply with the patent statutes, and to provide those skilled in the art with the information needed to apply the novel principles and to construct and use embodiments of the invention as required. It is to be understood, however, that various modifications can be accomplished without departing from the invention itself.

1-19. (canceled)

20. A method for suspending particles in a vessel, the method comprising:
(a) loading particles magnetically responsive to a magnetic field into a chamber defined by a vessel, and optionally loading a fluid volume into the chamber;
(b) applying a first magnetic field from one or more of a plurality of locations along a first path to effect a first magnetic response that urges a first rotational velocity to the particles;
(c) applying a second magnetic field from one or more of a plurality of locations along a second path to effect a second magnetic response that urges a second rotational velocity to the particles, wherein the second rotational velocity is generally opposed to the first rotational velocity; and
(d) prior to or subsequent to step (c), adjusting the first magnetic field to change the first magnetic response in the particles.

21. The method as in claim 20, including removing the first magnetic field prior to applying the second magnetic field.

22. The method as in claim 20, including applying the first magnetic field from a first side of the vessel, and applying the second magnetic field from a second side of the vessel, wherein the first side of the vessel is opposite from the second side of the vessel.

23. (canceled)

24. The method as in claim 21 wherein the first and second magnetic responses include attraction to draw the particles towards or against a respective wall in or defining the chamber.

25. (canceled)

26. The method as in claim 24, including removing at least a portion of the fluid volume from the chamber while at least one of the first and second magnetic fields are applied to the particles.

27. The method as in claim 26, including loading a second fluid volume into the chamber while the at least one of the first and second magnetic fields are applied to the particles.

28. The method as in claim 24 wherein the first and second magnetic fields are applied in a manner to substantially uniformly distribute and suspend the particles within the fluid volume.

29. (canceled)

30. The method as in claim 20 wherein the first path is arcuate and within a first plane that does not intersect with the chamber, and the second path is arcuate and within a second plane that does not intersect with the chamber.

31. The method as in claim 20 wherein the first magnetic response causes a third rotational velocity in the fluid, and the second magnetic response causes a fourth rotational velocity in the fluid, wherein the fourth rotational velocity is generally opposed to the third rotational velocity.

32. A method for suspending magnetically responsive particles in a fluid disposed in a chamber, the method comprising:
(a) providing a first movable magnetic field to which the particles are magnetically responsive, the first magnetic field being movable through the chamber and applicable from a first path that extends in proximity to a first side of the chamber;
(b) providing a second movable magnetic field to which that particles are magnetically responsive, the second magnetic field being movable through the chamber and applicable from a second path that extends in proximity to a second side of the chamber;
(c) applying the first and second magnetic fields in a series comprising:
(i) a first condition wherein the first magnetic field is applied to the particles while moving along the first path;
(ii) a second condition wherein the first magnetic field is modified in a manner to change the magnetic response in the particles or is not applied to the particles; and
(iii) a third condition wherein the second magnetic field is applied to the particles while moving along the second path

33. The method as in claim 32 wherein the second condition includes not applying the second magnetic field to the particles, the first condition includes not applying the second magnetic field to the particles, the second condition includes not applying the second magnetic field to the particles, and the third condition includes not applying the first magnetic field to the particles.

34. (canceled)

35. (canceled)

36. The method as in claim 32 wherein the series of applying the first and second magnetic fields proceeds in an order of the first condition followed by the second condition, and the second condition followed by the third condition.

37. The method as in claim 32 wherein the first and second magnetic fields are in continuous movement along their respective first and second paths throughout the series.

38. The method as in claim 33 wherein the first and second magnetic fields are movable with adjustable speed.

39. The method as in claim 32 wherein the first and second magnetic paths are arcuate.

40. The method as in claim 32 wherein at least one of the first and second magnetic fields are modifiable in field strength.

41-47. (canceled)

48. A method for suspending magnetically responsive particles in a fluid disposed in a chamber having an opening fluidically connected to a source for the fluid through a fluid channel, the method comprising:
(a) orienting the chamber so that the opening is gravitationally below the fluid and the particles in the chamber;
(b) deploying a gas bubble valve to selectively fluidically separate the chamber from the fluid channel, the gas bubble valve comprising a bubble containing gas;
(c) providing a first movable magnetic field to which the particles are magnetically responsive; and
(d) selectively moving the first movable magnetic field through the chamber to effect a magnetic response in the particles.

49. The method as in claim 48, including positioning the bubble at the opening to fill the opening.

50. (canceled)

51. The method as in claim 48, including
(i) removing the gas bubble valve to selectively fluidically connect the chamber to the fluid channel;
(ii) filling the chamber with a fluid volume through the opening while the gas bubble is removed; and
(iii) re-deploying the gas bubble valve subsequent to filling the chamber with the fluid volume.

52. (canceled)

53. (canceled)

54. The method as in claim 48 wherein the bubble contains air.
